(19) United States
(12) Patent Application Publication
(10) Pub. No.: US 2020/0100734 A1
(43) Pub. Date: Apr. 2, 2020

(54) MEDICAL MATTRESS AND METHOD FOR REAL-TIME MONITORING

(71) Applicant: Siemens Healthcare GmbH, Erlangen (DE)

(72) Inventor: Stefan Britzen, Buckenhof (DE)

(21) Appl. No.: 16/586,460

(22) Filed: Sep. 27, 2019

(30) Foreign Application Priority Data
Sep. 28, 2018 (EP) 18197560.8
Dec. 6, 2018 (EP) 18210735.9

(51) Int. Cl. A61B 6/04; A61N 5/10

(52) U.S. Cl. CPC A61B 6/0407; G06K 9/00342; A61N 5/1049

(57) ABSTRACT

A medical mattress for determining a position and/or a change in position of a patient supported on the mattress is provided. The mattress includes a number of fluid channels that are distributed in a matrix, such that a first part of the fluid channels extends parallel to each other and a second part of the fluid channels extends parallel to each other. The first part is orthogonal to the second part. Each of the fluid channels at an output is connected to a respective fluid supply hose. The mattress also includes a fluid supply device that pumps fluid into the fluid channels via the fluid supply hoses, sensors for measuring flow parameters and/or flow change parameters in the fluid channels, and an evaluation unit for evaluation of the flow parameters and/or flow change parameters with respect to a position and/or change in position of the patient supported on the mattress.

This application claims the benefit of EP 18197560.8, filed on Sep. 28, 2018, and EP 18210735.9, filed on Dec. 6, 2018, which are hereby incorporated by reference in their entirety.

### BACKGROUND

The present embodiments relate to a medical mattress for determining a position and a change in position of a patient supported on the mattress and to a method for real-time monitoring of positioning of the patient during a medical diagnosis and/or therapy.

During medical interventions on a patient, real-time X-ray images are taken for the navigation of medical instruments in the patient's body (e.g., in the head or heart) using fluoroscopic X-ray imaging (e.g., two-dimensional (2D) X-ray imaging). Compared to images from three-dimensional (3D) modalities (e.g., magnetic resonance imaging, computed tomography, or 3D angiography), these 2D X-ray images do not show spatial (3D) details. Instead, the 2D X-ray images are more readily available, more high-resolution, minimize the radiation exposure for patient and doctor, and depict interventional devices such as surgical instruments, catheters, guide wires, etc. in quasi-real time. Ideally, spatial information is recovered by registering and superimposing pre-operatively recorded volume images with the two-dimensional real-time X-ray images (e.g., 2D3D registration).

The combination of co-registered 2D and 3D images affords the doctor a very good orientation in the patient. It is desirable that such registration may be carried out as accurately as possible and without manual interaction. Any patient movements are to be registered and accordingly be reacted to by the system or the user. In the current clinical setup, patient movements are not automatically detected. The 2D3D registration is purely image-based and has to be initiated manually by the user.

The user must then check (e.g., manually check) whether the registration is still appropriate. After detected misregistration, the user then manually corrects the registration, resulting in inaccuracies and delays in the workflow. This is particularly disruptive in cyclically recurrent patient movements, such as breathing or the heartbeat. This creates constant misregistration and inaccuracy, especially in the region of the thorax or abdomen. In addition to interventions, the quality of radiotherapy in the case of lung and bronchial cancer also suffers from respiratory movements. There are various approaches for compensation, but none of them work reliably and may all potentially damage healthy tissue as well during radiotherapy.

The following solutions for monitoring patient position and changes in position are known from the prior art: (a) A registration of the volume to the C-arm with “optical surface detection,” for example, by 3D cameras with a view of the patient; however, the problem with these methods is that the methods demand a clear view of the patient, which is hardly possible, especially in surgeries due to the large amount of equipment; (b) Various methods for 2D3D or 3D3D registration; (c) a detection of breathing by strain detectors that are fixed around the chest and therefore detect the breathing movement; the disadvantage lies in the limited type of one-dimensional motion detection, which does not adequately capture the actual complexity of breathing; (d) in radiotherapy, invasive markers (e.g., fiducials) in the vicinity of or in the tumor, as well as parallel kV imaging, are used to track the target; both techniques have dangers and limitations (e.g., pneumothorax when setting the fiducials, or non-visibility of the tumor with kV imaging) that provide an increased risk for the patient; (e) terahertz cameras or similar sensors (e.g., in the use of airport security).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an alternative device that allows simple and reliable monitoring of positions and changes in position of a patient during an operation with fluoroscopic imaging is provided. As another example, a method for real-time monitoring of a patient during a medical diagnosis and/or therapy is provided.

A medical mattress of one or more of the present embodiments for determining the position and/or change in position of a patient supported on the mattress is at least partially made of X-ray permeable materials. The medical mattress has a top configured as a lying surface for the patient, and a bottom opposing the top. The medical mattress also has four edge sides opposing each other in pairs. The four edge sides connect the top and the bottom. The medical mattress includes a large number of fluid channels that are arranged distributed over at least a portion of the mattress between the top and the bottom in a matrix, such that a first part of the fluid channels extends parallel to each other from a first edge side to a second opposing edge side through the mattress and a second part of the fluid channels extends parallel to each other from a third edge side to an opposing fourth edge side through the mattress. The first part is orthogonal to the second part. Each of the number of fluid channels at a respective output is connected to a fluid supply hose. The medical mattress includes a fluid supply device that pumps fluid into the fluid channels via the fluid supply hoses, and sensors for measuring flow parameters and/or flow change parameters and/or pressure change parameters in the fluid channels. The medical mattress also includes an assigned evaluation unit for the evaluation of the flow parameters and/or flow change parameters and/or pressure change parameters measured by the sensors with respect to a position and/or change in position of the patient supported on the mattress.

Using the medical mattress of one or more of the present embodiments, monitoring of a patient with respect to a change in position or movement may be carried out in a simple way during a medical diagnosis and/or therapy procedure with an imaging device, so that further acts may be taken (e.g., with regard to a warning or a notice or a re-registration). The mattress may provide information for or about position profiles and changes in position quickly and in real time. A mattress of this kind has the advantage that the mattress may be made completely of X-ray transparent materials in the examination area, and therefore, no restrictions regarding imaging are to be expected. The matrix of air ducts is, for example, adapted to the required measurement accuracy in terms of granularity. The evaluation unit may also be arranged outside the mattress and only has a communication link with the mattress. Flow parameters and/or flow change parameters may be measured values from which flows or flow changes may be determined (e.g., both direct flow and flow change measurements; also other measured values, from which flows or flow changes may be calculated). For example, pressures or pressure changes may also be measured therefore.

According to one embodiment, the fluid channels are formed by air ducts, the fluid supply hoses are formed by air supply hoses, and the fluid supply device is formed by an air supply device. “Air” is in this case may be taken to be a gas that may be inhaled in relatively large quantities without being harmful to a patient. Air is readily available and easy to use for the mattress. The corresponding air supply hoses and air supply devices may also be used easily and quickly.

According to a further embodiment, the sensors are formed by flow sensors. Such sensors may detect air flows and pressure changes of the respective assigned fluid channel or air duct of the mattress and thereby provide the information necessary to create an exact position profile or changes in position of the patient supported on the mattress. Pressure sensors may also be used, for example, if a defined resistance is applied to the output in front of the sensor of the channel, and therefore, the pressure drop corresponds to the increased resistance in the mattress.

According to a further embodiment, the mattress is partially (e.g., in the air ducts) formed from an air permeable material (e.g., foam). In this way, the mattress may be compressed easily and reliably, so that the mattress is compressed by a position-dependent pressure (e.g., patient weight) acting thereon accordingly and a pressure drop in the correspondingly compressed air ducts that generates flow changes. Foam materials are suitable for such use and are proven for use in mattresses. Foams are also X-ray transparent or at least mostly X-ray transparent.

For example, the fluid channels or air ducts are implemented in that webs are arranged between the fluid channels. The webs may also be X-ray transparent and compressible. The webs may also be air-impermeable. The orthogonal structure of the matrix of fluid channels or air ducts is easily implemented via the webs, which are inserted at fixed positions.

According to a further embodiment, the evaluation unit is configured for the creation of a position profile of the patient (e.g., using machine learning methods). For this purpose, for example, an algorithm or software may be used to calculate such a position profile from the flow parameters. Algorithms already trained with a large number of data sets may be used within the context of machine learning or deep learning process.

According to a further embodiment, the evaluation unit is configured for real-time monitoring of changes in position of the patient (e.g., using machine learning methods). Flow change parameters registered by the sensors may also be processed by an algorithm or software to provide information about a change in position or movement of the patient. The entire processing chain may be carried out so quickly that even the smallest movements or short and regular movements are registered. Algorithms already trained with a large number of data sets may be used within the context of a machine learning or deep learning process.

In one embodiment, the mattress may also have a storage unit for storing data and measured values in order to store the parameters recorded by the sensors or the evaluated position profiles and changes in position. The information may also be passed on to other devices, such as an imaging device.

According to a further embodiment, the air supply device is formed by a pump or a compressor.

According to a further embodiment, the sensors for measuring flow and/or flow change parameters are arranged on or in the fluid supply hoses or air supply hoses. In this way, it is easy to arrange non-X-ray transparent parts, which the sensors usually contain, outside a possible X-ray screening range, while at the same time accurately measuring the flow conditions. In this context, the fluid supply hoses or air supply hoses may be arranged outside the X-ray beam screening area (e.g., to the side of the mattress), so that the fluid supply hoses or air supply hoses may be combined. In this way, the fluid supply hoses or air supply hoses may not only be kept out of the screening area but may also be bundled so as to be compact and stumble-proof.

According to a further embodiment, the matrix of fluid channels extends over at least 50% of the mattress. In this way, even small changes in position and movements of the patient may be reliably registered.

According to a further embodiment, the mattress has a device for heating the fluid. In this way, temperature-controlled ventilation may also be provided for warming of the patient. The patient weight and/or patient size may also be obtained from the flow and/or flow change parameters using a calibration.

The method of one or more of the present embodiments for real-time monitoring of the positioning of a patient during a medical diagnosis and/or therapy with a medical imaging device using a mattress of the present embodiments, on which the patient is positioned, has the following acts: pumping fluid (e.g., air) into the fluid channels (e.g., air ducts) through the fluid supply device (e.g., air supply device) via the fluid supply hoses (e.g., air supply hoses); measuring flow and/or flow change parameters in the fluid channels (e.g., air ducts); and evaluating the flow and/or flow change parameters measured by the sensors with respect to a position and/or change in position of the patient. The acts are carried out continuously and/or at predetermined time intervals up to a cancellation criterion. Pumping may be carried out continuously, while the measurement and evaluation is carried out continuously or at predetermined time intervals if necessary.

According to an embodiment, a position profile is created from the measured flow and/or flow change parameters and used for a registration of the medical imaging device with the patient position. In this way, a registration of the imaging device may be carried out with previously recorded volume images.

According to a further embodiment, a notice or warning is passed on if the evaluation results in a change in position of the patient. Either a warning signal may be issued to a user, or automatic triggering of an action adapted to the situation may be performed. Therefore, a re-registration of the medical imaging device with the patient position may be triggered if the evaluation results in a change in position of the patient. This provides continuous real-time monitoring of patient movements with automatic registration correction, providing optimum imaging. During medical interventions, medical staff may fully focus on the procedure, reducing the error rate and promoting improved patient care.

### BRIEF DESCRIPTION OF THE DRAWINGS

FIG. 1 shows a view of one embodiment of a medical mattress;

FIG. 2 shows a view of an exemplary arrangement matrix for air ducts of a medical mattress;

FIG. 3 shows an enlarged cross-section through one embodiment of a medical mattress;

FIG. 4 shows one embodiment of a medical imaging device with a medical mattress;

FIG. 5 shows a view of an exemplary position profile; and

FIG. 6 shows a sequence of an embodiment of a method.

### DETAILED DESCRIPTION

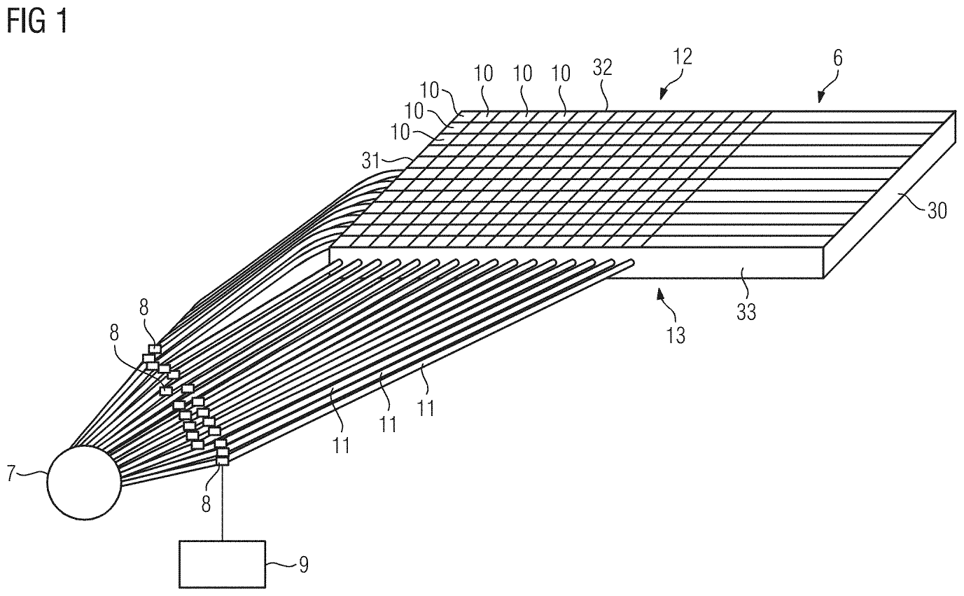

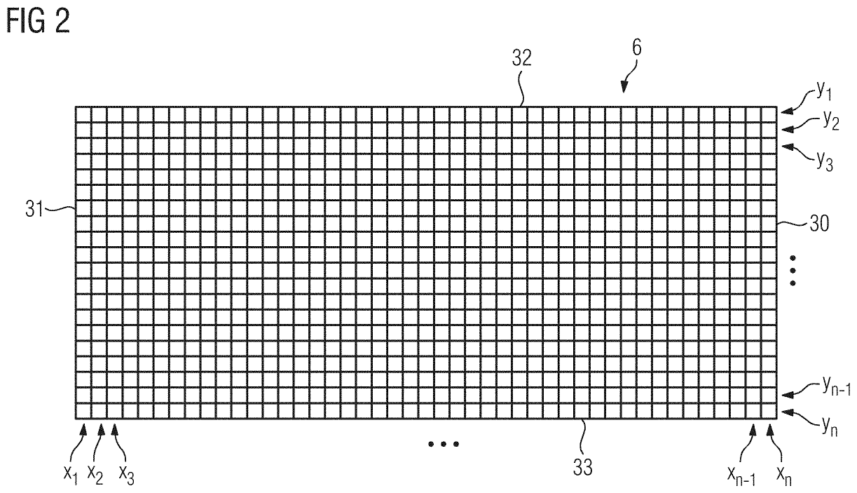

FIGS. 1 and 2 show one embodiment of a medical mattress 6 with a top 12 for supporting a patient. The medical mattress 6 has a large number of air ducts 10 that extend matrix-like through the mattress. Air ducts of a first part (y₁, y₂, . . . , yₙ₋₁, yₙ) of the air ducts are parallel to each other and extend from a first edge side 30 to a second edge side 31, air ducts of a second part (x₁, x₂, . . . , xₙ₋₁,xₙ) of the air ducts are also parallel to each other and extend from a third edge side 32 to a fourth edge side 33. The second part (x₁, x₂, . . . , xₙ₋₁i, xₙ) of the air ducts and the first part (y₁, y₂, . . . , yₙ₋₁, yₙ) of the air ducts are orthogonal to each other, so that a matrix results from this. The matrix may only partially cover the mattress 6, as shown in FIG. 1, or cover the entire mattress 6, as shown in FIG. 2. The portion over which the matrix extends occupies, for example, at least 50% of a surface of a top of the mattress. The top 12 of the mattress 6 may have a covering or a cover.

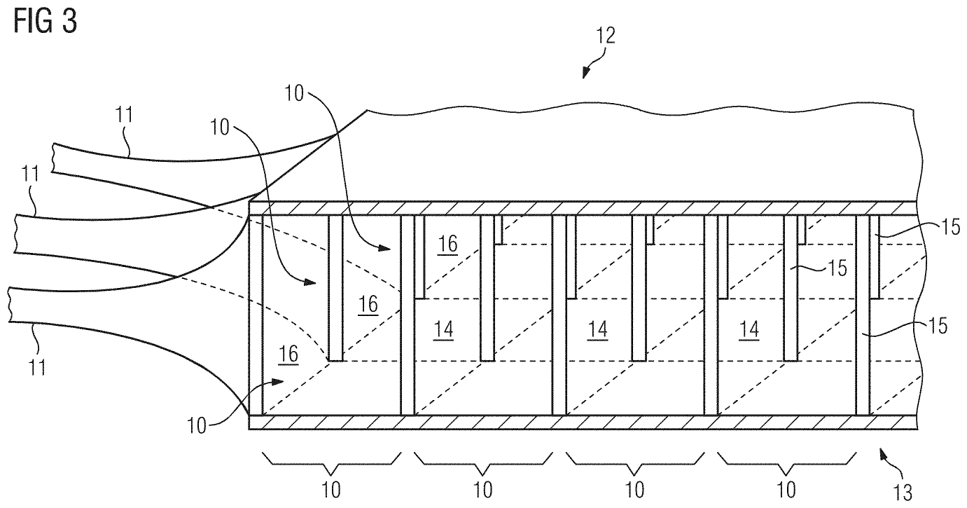

Each of the air ducts 10 is connected at an output 16 to an air supply hose 11, as shown in FIG. 1 and enlarged also in the cross-section of FIG. 3. The top 12, the bottom 13, as well as the edge sides, to which no air supply hoses are connected, may be sealed in an airtight manner, so that air that is pumped through the air supply hose 11 into the air duct cannot escape. Using an air supply device 7 (e.g., a compressor or a pump), air is pressed through the air supply hoses into the air ducts 10. In addition, sensors 8 are provided for the measurement of flow and/or flow change parameters and/or pressure change parameters of the air ducts 10. Sensors 8 measure the respective parameters either continuously or at specified intervals.

The air ducts 10 are also X-ray transparent, as are the air supply hoses 11. The material from which the mattress is formed in the air ducts 10 is air permeable and elastically formed to make compression of the mattress possible. The material may be, for example, a foam 14 (e.g., an elastomeric foam (polyurethane soft foam, acrylonitrile butadiene rubber) or a thermoplastic foam (PS-E, PP-E, PVC-E)) that is also X-ray transparent. The mattress is configured to be X-ray transparent (e.g., in the possible X-ray screening area). Between the air ducts, webs 15 that are also compressible and X-ray transparent may be arranged. The orthogonal structure of the matrix is implemented via the webs 15.

Sensors 8, unless not also X-ray transparent, are arranged in such a way that the sensors 8 are or may be arranged outside the X-ray beam screening area. If the sensors 8 are mounted, for example, at the end of the air supply hoses, the air supply hoses may be bundled. Then, the sensors 8 may be arranged on the side of the mattress (right or left of the mattress or on both sides) or in the foot area of a patient couch on which the medical mattress rests, so that during an X-ray examination, the sensors 8 are located below the X-ray detector. The sensors 8 may also be arranged in the region of the output 16 of the air ducts.

Due to different mattress loading on the part of a patient arranged on the mattress at different points of the mattress, the air-permeable structure is compressed to a greater or lesser extent, resulting in a pressure drop corresponding to the loading above the respective air duct. The pressure drop is then measured by the respective sensor 8. From the parameters, a position profile of the patient or changes in position or movements of the patient may then be determined by an evaluation unit 9 and, for example, by software or an algorithm. Machine learning or deep learning algorithms that have already been trained with a large number of data sets of subjects may also be used for this purpose in order to identify the actual position or movement of the patient from the patient's characteristic patterns. Movements may then be interpreted, for example, as breathing or heart movements or other movements. The result of the evaluation of the flow and/or flow change parameters supplied by the sensors may be, for example, a position profile 19, as shown in FIG. 5. Accuracy depends on the dimension of the matrix of air ducts.

Due to the medical mattress 6, effective real-time monitoring of the patient's position may be carried out without additional camera technology. The initial position of the patient as well as real-time changes or movements or displacements may be detected. For example, regular pressure fluctuations in the patient's thoracic and abdominal areas may be interpreted as a breathing movement. When using the mattress during X-rays, therapies, and interventional procedures, optimum patient care is thus possible.

The mattress 6 may also have a device for heating air. In this way, temperature-controlled ventilation may also be provided for warming the patient. The patient's weight and/or patient's size may also be obtained from the flow and/or flow change parameters using a calibration.

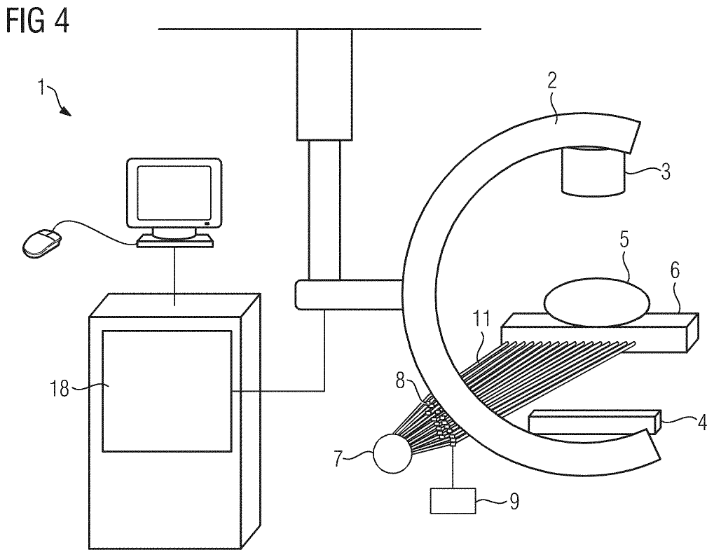

FIG. 4 shows one embodiment of a medical imaging device 1 that X-rays the patient 5 arranged on the mattress. The medical imaging device 1 has, for example, a movable C-arm 2 with an X-ray source 3 and an X-ray detector 4, and is controlled by a system control 18. The sensors 8 of the mattress and the air supply device 7 are located on the side of the mattress outside the X-ray beam screening area of the X-ray source 3. Not shown is a patient couch on which the mattress and the patient are supported. The sensors of the mattress and the air supply device 7 may also be arranged in a foot area of the patient couch. The evaluation unit 9 of the mattress may also have only a communications link with the mattress and have the measurement data or parameters transmitted by the sensors. The evaluation unit may, for example, also be part of the medical imaging device 1.

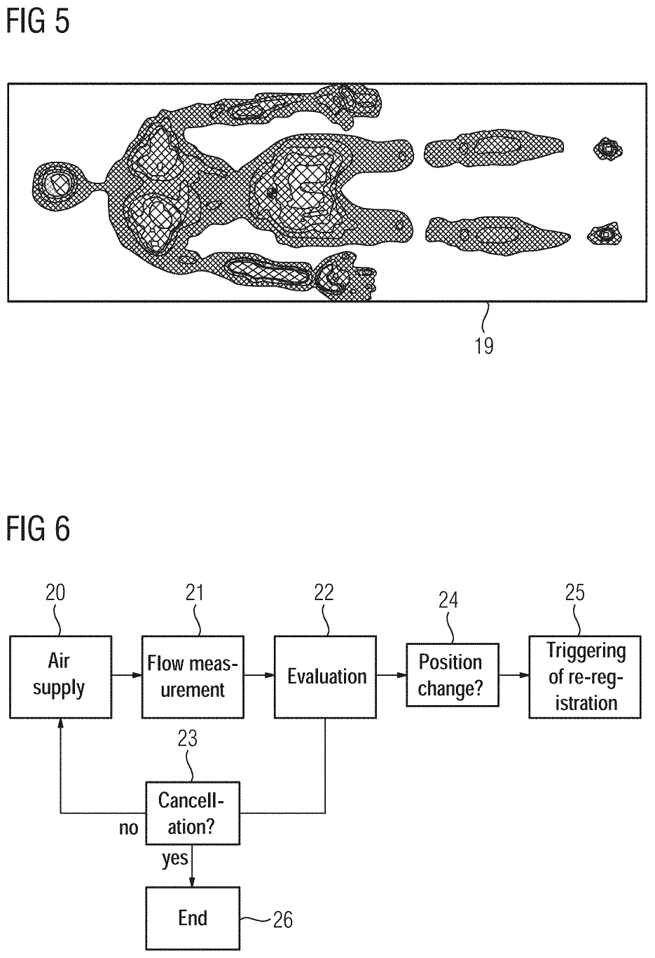

FIG. 6 shows a procedure of an embodiment of a method for real-time monitoring of a patient's positioning during a medical diagnosis and/or therapy with a medical imaging device using the described mattress, on which the patient is supported. In a first act 20, air is pumped into the air ducts via the air supply hoses by the air supply device. The mattress is compressed by a patient supported on a top of the mattress according to the weight of the patient, and the air ducts are compressed accordingly. As a result, the flow conditions in the respective air ducts change compared to non-compressed air ducts. In a second act 21, flow parameters and/or flow change parameters are measured in the air ducts. These are then evaluated by the evaluation unit 9 in a third act 22, and, more precisely, with regard to a position and/or change in position of the patient.

For example, a position profile of the patient may be created from the parameters (e.g., using software and/or a pre-trained machine learning algorithm). The position profile thus created may be used, for example, for a registration of the patient position with the imaging device or with previously recorded volume images of the patient. As soon as a cancellation criterion 23 applies (e.g., a previously set time or a user input or similar), the method comes to an end 26. If no cancellation criterion 23 applies, the method is continued. While the air supply takes place, for example, continuously, the measurement of the second act 21 and the evaluation of the third act 22 may be carried out continuously or at predetermined time intervals, depending on the need or preset.

Position changes may also be evaluated from the parameters if the patient slips or changes his position due to cyclic or spontaneous movements. Changes in position may be evaluated individually, or a new position profile may be created and compared with the previous position profile to derive changes in position. All this may be done live and in real time if necessary (e.g., during a fluoroscopic examination, as part of an operation, or an interventional procedure). As soon as a cancellation criterion 23 applies (e.g., a previously set time or a user input or similar), the method comes to an end 26.

In addition, as soon as a change in position is queried and detected in an optional fourth act 24, a notice or warning may be passed on. Alternatively, in an optional fifth act 25, a re-registration of the medical imaging device with the patient position may also be triggered.

The mattress of one or more of the present embodiments has a large number of advantages over the prior art: an initial registration of the patient's position may be carried out automatically based on information from the mattress (and the system position of the imaging device) with existing 3D volume images. Live monitoring of patient movements may be performed and used as a warning for medical personnel or the imaging device. As a result, an existing registration may be automatically corrected. Regular pressure fluctuations in the patient's thoracic and abdominal areas may be evaluated as respiratory triggering. Further monitoring devices in the intervention space are not necessary or may be omitted (e.g., cameras). The mattress does not have image-disturbing electronics in the X-ray screening area, so that the imaging is of unaffected high quality. Optionally, temperature-controlled ventilation may be implemented through the mattress to heat the patient during the examination. In addition, the patient's weight and size may be derived from data (e.g., calibrated data).

In addition to the use of air as a fluid, other gases or liquids may also be used (e.g., water).

One or more of the present embodiments may be briefly summarized as follows: A medical mattress for determining the position and/or change in position of a patient supported on the mattress. The mattress is at least partially made of X-ray permeable materials. The mattress has a top that is configured as a lying surface for the patient, a bottom opposing the top, and four edge sides opposing each other in pairs. The four edge sides connect the top and the bottom. The mattress includes a large number of fluid channels that are arranged distributed over at least a portion of the mattress between the top and the bottom in a matrix, such that a first part of the fluid channels extends parallel to each other from a first edge side to a second opposing edge side through the mattress and a second part of the fluid channels extends parallel to each other from a third edge side to an opposing fourth edge side through the mattress. The first part is orthogonal to the second part. Each of the number of fluid channel at a respective output is connected to a fluid supply hose. The mattress also includes a fluid supply device that pumps fluid into the fluid channels via the fluid supply hoses. The mattress includes sensors for measuring flow parameters and/or flow change parameters in the fluid channels, and an assigned evaluation unit for the evaluation of the flow parameters and/or flow change parameters measured by the sensors with respect to a position and/or change in position of the patient supported on the mattress.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical mattress for determining a position, a change in position, or the position and the change in position of a patient supported on the medical mattress, the medical mattress being at least partially made of X-ray permeable materials, the medical mattress having a top that is configured as a lying surface for the patient, a bottom opposing the top, and four edge sides opposing each other in pairs, the four edge sides connecting the top and the bottom, the medical mattress comprising:
a number of fluid channels that are arranged distributed over at least a portion of the medical mattress between the top and the bottom in a matrix, such that a first part of the number of fluid channels extends parallel to each other from a first of the four edge sides to a second opposing edge side of the four edge sides through the medical mattress and a second part of the number of fluid channels extends parallel to each other from a third of the four edge sides to an opposing fourth edge side of the four edges sides through the medical mattress, wherein the first part is orthogonal to the second part, and wherein each fluid channel of the number of fluid channels at a respective output is connected to a fluid supply hose;
a fluid supply device configured to pump fluid into the number of fluid channels via the fluid supply hoses;
sensors configured to measure flow, flow change, or flow and flow change parameters, pressure change parameters, or any combination thereof in the number of fluid channels; and
an assigned evaluation unit configured to evaluate the flow parameters, the flow change parameters, the pressure change parameters, or the respective combination thereof measured by the sensors with respect to the position, the change in position of the patient supported on the medical mattress, or a combination thereof.

2. The medical mattress of claim 1, wherein the number of fluid channels are formed by air ducts, the fluid supply hoses are formed by air supply hoses, and the fluid supply device is formed by an air supply device.

3. The medical mattress of claim 1, wherein the sensors are formed by flow sensors or pressure sensors.

4. The medical mattress of claim 2, wherein the medical mattress is partially formed from an air permeable material.

5. The medical mattress of claim 4, wherein the air ducts are formed from the air permeable material.

6. The medical mattress of claim 4, wherein the air permeable material is a foam.

7. The medical mattress of claim 1, wherein the assigned evaluation unit is configured to create a position profile of the patient using machine learning methods.

8. The medical mattress of claim 1, wherein the assigned evaluation unit is configured for real-time monitoring of changes in position of the patient.

9. The medical mattress of claim 1, further comprising a memory configured to store data and measured values.

10. The medical mattress of claim 2, wherein the air supply device is formed by a pump or a compressor.

11. The medical mattress of claim 2, wherein the sensors are arranged on or in the air supply hoses.

12. The medical mattress of claim 1, wherein the sensors are arrangeable outside an X-ray beam irradiating the patient.

13. The medical mattress of claim 11, wherein the air supply hoses are arranged outside an X-ray beam screening area, so that the air supply hoses are combinable.

14. The medical mattress of claim 13, wherein the air supply hoses are arranged to the side of the medical mattress.

15. The medical mattress of claim 1, wherein the matrix of the number of fluid channels extends over at least 50% of the medical mattress.

16. The medical mattress of claim 1, further comprising webs between the number of fluid channels,
wherein the number of webs are X-ray transparent and compressible.

17. The medical mattress of claim 1, further comprising a device configured for heating the fluid.

18. A method for real-time monitoring of positioning of a patient during a medical diagnosis, therapy, or diagnosis and therapy with a medical imaging device using a mattress, on which the patient is supported, for determining a position, a change in position, or the position and the change in position of the patient supported on the mattress, the mattress being at least partially made of X-ray permeable materials, the mattress having a top that is configured as a lying surface for the patient, a bottom opposing the top, and four edge sides opposing each other in pairs, the four edge sides connecting the top and the bottom, the mattress comprising a number of fluid channels that are arranged distributed over at least a portion of the mattress between the top and the bottom in a matrix, such that a first part of the number of fluid channels extends parallel to each other from a first of the four edge sides to a second opposing edge side of the four edge sides through the mattress and a second part of the number of fluid channels extends parallel to each other from a third of the four edge sides to an opposing fourth edge side of the four edges sides through the mattress, wherein the first part is orthogonal to the second part, and wherein each fluid channel of the number of fluid channels at a respective output is connected to a fluid supply hose, the mattress further comprising a fluid supply device configured to pump fluid into the number of fluid channels via the fluid supply hoses, sensors configured to measure flow, flow change, or flow and flow change parameters, pressure change parameters, or any combination thereof in the number of fluid channels, and an assigned evaluation unit configured to evaluate the flow parameters, the flow change parameters, the pressure change parameters, or the respective combination thereof measured by the sensors with respect to the position, the change in position of the patient supported on the medical mattress, or a combination thereof, the method comprising:
pumping fluid into the number of fluid channels through the fluid supply device via the fluid supply hoses;
measuring the flow, flow change, or flow and flow change parameters, the pressure change parameters, or the respective combination thereof in the number of fluid channels; and
evaluating the flow, flow change, or flow and flow change parameters, the pressure change parameters, or the respective combination thereof measured by the sensors with respect to the position, the change in position of the patient, of the combination thereof,
wherein the pumping, the measuring, and the evaluating are carried out continuously, at predetermined time intervals, or continuously and at the predetermined time intervals, respectively, up to a cancellation criterion.

19. The method of claim 18, wherein measuring the flow, flow change, or flow and flow change parameters, the pressure change parameters, or the respective combination thereof comprises measuring the flow, flow change, or flow and flow change parameters,
wherein the method further comprises:
creating a position profile from the measured flow, flow change, or flow and flow change parameters; and
using the created position profile for a registration of the medical imaging device with the patient position.

20. The method of claim 18, further comprising passing on a notice or warning when the evaluation results in a change in position of the patient.

21. The method of claim 18, further comprising triggering a re-registration of the medical imaging device with the patient position when the evaluation results in a change in position of the patient.
